(19) United States
(12) Patent Application Publication
(10) Pub. No.: US 2015/0174052 A1
(43) Pub. Date: Jun. 25, 2015

(54) HAIR CARE PRODUCTS WITH ENHANCED CARE PERFORMANCE

(71) Applicant: Henkel AG & Co. KGaA, Duesseldorf (DE)

(72) Inventors: Manuela Mette, Kleinfeld (DE); Thomas Hippe, Appen (DE); Erik Schulze zur Wiesche, Hamburg (DE); Monika Nebel, Norderstedt (DE)

(21) Appl. No.: 14/636,904

(22) Filed: Mar. 3, 2015

Related U.S. Application Data
(63) Continuation of application No. PCT/EP2013/066033, filed on Jul. 30, 2013.

(30) Foreign Application Priority Data
Sep. 6, 2012 (DE) 10 2012 215 799.9

(51) Int. Cl. A61K 8/898; A61K 8/42; A61K 8/67; A61K 8/92; A61K 8/81; A61Q 5/12; A61K 8/60; A61K 8/41

(52) U.S. Cl. CPC A61K 8/898; A61K 8/602; A61K 8/42; A61K 8/416; A61K 8/92; A61K 8/8152; A61Q 5/12; A61K 8/673

(57) ABSTRACT

Compositions for treating keratin fibers, in particular human hair, that significantly improving the conditioning properties, shine, and haptics include as essential ingredients cationic ammonia compounds, amidoamines, ester oils, cationic polymers, a selected amodimethicone, and sugar surfactants.

### FIELD OF THE INVENTION

The present invention generally relates to compositions for the treatment of keratinic fibers, in particular human hair, which appreciably improve the conditioning properties as well as the shine and haptics of keratinic fibers, in particular human hair.

### BACKGROUND OF THE INVENTION

The cosmetic treatment of keratinic fibers, in particular of human hair, is an important element in human personal care. Human hair is therefore nowadays treated with hair-cosmetic preparations in many ways.

A demand exists for active agents or active-agent combinations for cosmetic agents having good care-providing properties and good biodegradability. The compatibility of the cosmetic compositions is furthermore an extraordinarily important criterion.

Compositions for utilization on keratinic fibers, in particular human hair, not only must exhibit good cleaning and conditioning and care-providing capabilities, but furthermore should be highly compatible and, even when used frequently, should not result in severe degreasing or dryness and splitting. The feel of the keratinic fibers after cosmetic treatments is an essential criterion as to whether the corresponding composition is perceived by the consumer as pleasant. The sensory properties and especially the haptics of a composition are thus essential effects that the consumer can experience. What is desired is therefore precisely those compositions which not only provide care to the keratinic fibers, in particular human hair, but furthermore, by way of their sensory and in particular haptic properties, perceptibly and tangibly modify the surface of keratinic fibers, in particular human hair. The compositions are moreover intended to regenerate and balance the structure especially in the interior of the keratinic fibers, in particular the human hair. At the same time, the compositions are to be simple and economical to produce.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic composition for conditioning keratinic fibers, including in a cosmetic carrier a) 0.1 to 6.0 wt % of at least one cationic surfactant selected from behentrimonium chloride and/or dimethyldibehenylammonium chloride and/or tribehenylmethyltrimonium chloride and/or cetyltrimethylammonium chloride and/or dicetyldimethylammonium chloride and/or tricetylmethylammonium chloride and/or trimethylstearylammonium chloride, dimethyldistearylammonium chloride, and/or tristearylmethylammonium chloride, as well as mixtures thereof; b) 0.01 to 6.0 wt % of at least one amidoamine and/or one permanently cationic amidoamine; c) 0.01 to 7.5 wt % of at least one ester oil; d) 0.01 to 5.0 wt % of at least one cationic polymer selected from Polyquatemium-67 and/or Polyquatemium-72 and/or Polyquaternium-74 and/or Polyquaternium-37; and e) 0.01 to 10.0 wt % of at least one amodimethicone selected from amodimethicones having the INCI name Amodimethicone/Morpholinomethyl Silsesquioxane Copolymer; f) 0.001 to 3.0 wt % of at least one sugar surfactant; all quantity indications being based on the total weight of the cosmetic composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that the above-recited requirements are meet, completely surprisingly, by a cosmetic composition that includes, in a cosmetic carrier,
a) 0.1 to 6.0 wt % of at least one cationic surfactant selected from behentrimonium chloride and/or dimethyldibehenylammonium chloride and/or tribehenylmethyltrimonium chloride and/or cetyltrimethylammonium chloride and/or dicetyldimethylammonium chloride and/or tricetylmethylammonium chloride and/or trimethylstearylammonium chloride, dimethyldistearylammonium chloride, and/or tristearylmethylammonium chloride, as well as mixtures thereof,
b) 0.01 to 6.0 wt % of at least one amidoamine and/or one permanently cationic amidoamine,
c) 0.01 to 7.5 wt % of at least one ester oil,
d) 0.01 to 5.0 wt % of at least one cationic polymer selected from Polyquaternium-67 and/or Polyquaternium-72 and/or Polyquaternium-74 and/or Polyquaternium-37,
e) 0.01 to 10.0 wt % of at least one amodimethicone selected from amodimethicones having the INCI name Amodimethicone/Morpholinomethyl Silsesquioxane Copolymer, and
f) 0.001 to 3.0 wt % of at least one sugar surfactant,
   all quantity indications being based on the total weight of the cosmetic composition.

The use of this combination results in surprisingly good properties for the treated hair, in particular in improved combability values, improved shine, and improved elasticity, as well as an appreciable increase in the washing resistance of colored hair, and in longer durability simultaneously with better reshaping performance in the context of waving operations such as water waving and permanent waving.

“Hair treatment agents” for purposes of the present invention are, for example, hair shampoos, hair conditioners, conditioning shampoos, hair rinses, hair therapies, hair packs, hair tonics, hair coloring shampoos, or combinations thereof. Compositions that condition the hair, such as hair rinses, hair therapies, hair packs, hair oils and lotions, both as leave-on products, i.e. products that remain on the hair until it is next washed, and as rinse-off products, i.e. products to be rinsed off again a few seconds to a few hours after application, are to understood in particular as being among the hair treatment agents according to the present invention.

“Combability” is understood according to the present invention as both the combability of the wet fibers and the combability of the dry fibers.

“Softness” is defined as the tactility of an assemblage of fibers, in which context one skilled in the art sensorially feels and evaluates the “fullness” and “suppleness” parameters of the assemblage.

“Shapability” is understood as the ability to impart a change in shape to an assemblage of previously treated keratin-containing fibers, in particular human hairs. The term “stylability” is also used in hair cosmetics.

“Restructuring” is to be understood for purposes of the invention as a reduction in the damage to keratinic fibers resulting from a wide variety of influences. Restoration of natural strength plays an essential role here, for example. Restructured fibers are notable for improved shine, improved softness, and easier combability. In addition, they exhibit improved strength and elasticity. Successful restructuring can moreover be demonstrated physically as an increase in melting point as compared with the damaged fiber. The higher the melting point of the hair, the stronger the structure of the fiber.

“Washing fastness” is to be understood for purposes of the invention as maintenance of the original coloring, in terms of shade and/or intensity, when the keratinic fiber is exposed to the repeated influence of aqueous agents, in particular surfactant-containing agents such as shampoos.

The compositions according to the present invention that include the active-agent complex according to the present invention are furthermore notable for an appreciably improved condition of the keratinic fibers in terms of the moisture budget of the keratinic fibers. The active-agent complex according to the present invention furthermore results in appreciable protection of the keratinic fibers from heat effects, for example when blow-drying keratinic fibers. Protection of the surface of keratinic fibers from heat effects is very important especially when straightening irons or hair dryers are used. Lastly, it has been found, surprisingly, that the compositions according to the present invention result in appreciably delayed re-soiling of the keratinic fibers.

An “aqueous” cosmetic carrier includes at least 50 wt % water.

“Aqueous alcoholic” cosmetic carriers are to be understood for purposes of the present invention as aqueous solutions that include 3 to 70 wt % of a C₁ to C₆ alcohol, in particular methanol, ethanol, or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol, or 1,6-hexanediol. The agents according to the present invention can additionally include further organic solvents, for example methoxybutanol, benzyl alcohol, ethyl diglycol, or 1,2-propylene glycol. All water-soluble organic solvents are preferred in this context. Water is particularly preferred.

Cationic surfactants of formula (Tkat1) are the first group of obligatory ingredients of the compositions according to the present invention.

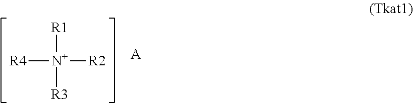

In formula (Tkat1), R1, R2, R3, and R4, mutually independently in each case, generally denote hydrogen, a methyl group, a saturated, branched or unbranched alkyl residue having a chain length from 8 to 30 carbon atoms, which optionally can be substituted with one or more hydroxy groups and “A” denotes a physiologically acceptable anion, for example halides such as chloride or bromide, as well as methosulfates.

Compounds in accordance with the invention are behentrimonium chloride and/or dimethyldibehenylammonium chloride and/or tribehenylmethyltrimonium chloride and/or cocotrimonium chloride and/or dicocodimonium chlorides, and/or tricocomonium chlorides and/or cetyltrimethylammonium chloride and/or dicetyldimethylammonium chloride and/or tricetylmethylammonium chloride and/or trimethylstearylammonium chloride, dimethyldistearylammonium chloride and/or stearyltrimethylammonium chloride and/or mixtures thereof. Preferred compounds are behentrimonium chloride and/or dimethyldibehenylammonium chloride and/or cetrimonium chloride and/or dicetyldimethylammonium chloride and/or stearyltrimethylammonium chloride and/or dimethyldistearylammonium chloride and/or mixtures thereof. Behentrimonium chloride and/or cetyltrimethylammonium chloride and/or stearyltrimethylammonium chloride and/or mixtures thereof are particularly preferred. Behentrimonium chloride and/or cetyltrimonium chloride, as well as a mixture thereof, are highly preferred.

The cationic surfactants according to the present invention are included in the compositions according to the present invention in a quantity from 0.1 to 6.0 wt % based on the total weight of the cosmetic preparations. Quantities from 0.2 to 4.5 wt % are preferred. Quantities from 0.2 to 3.0 wt % are more preferred, and quantities from 0.3 to 2.5 are highly preferred, based in each case on the total weight of the cosmetic preparation.

The second group of essential ingredients of the present invention are amidoamines and/or permanently cationized amidoamines, having in particular the following structural formulas:

R1-NH—(CH₂)ₙ—N⁺R²R³R⁴A (Tkat3),

in which R1 signifies an acyl or alkyl residue having respectively 6 to 30 carbon atoms which can respectively be branched or unbranched, saturated or unsaturated, and wherein the acyl residue and/or the alkyl residue can include at least one OH group, and R2, R3, and R4, mutually independently in each case, signify
1) hydrogen, or
2) an alkyl residue having 1 to 4 carbon atoms, which can be identical or different, saturated or unsaturated, and
3) a branched or unbranched hydroxyalkyl group having one to 4 carbon atoms, having at least one and at most three hydroxy groups, for example CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH, and hydroxybutyl residues, and
   A signifies a physiologically acceptable anion, and
   n signifies an integer between 1 and 10.

Halide ions, sulfate ions, phosphate ions, methosulfate ions, as well as organic ions such as lactate, citrate, tartrate, and acetate ions are appropriate, for example, as physiologically acceptable counter ions A. Methosulfates and halide ions, in particular chloride, are preferred.

A composition in which the amidoamine and/or the quatemized amidoamine according to the general formulas (Tkat3) is an amidoamine and/or a quaternized amidoamine, in which R1 signifies a branched or unbranched, saturated or unsaturated acyl residue having 6 to 30 carbon atoms, which can include at least one OH group, is preferred.

A fatty acid residue made of oils and waxes, in particular of natural oils and waxes, is preferred here. Suitable examples thereof are lanolin, beeswax, or candelilla wax.

Also preferred are those amidoamines and/or quaternized amidoamines in which R2, R3, and/or R4 in formula (Tkat3) signify a residue according to the general formula CH₂CH₂OR5, in which R5 can have the meaning of alkyl residues having 1 to 4 carbon atoms, hydroxyethyl, or hydrogen. The preferred value of n in the general formula (Tkat3) is an integer between 2 and 5.

The alkylamidoamines both can be present as such, and can be converted by protonation in a correspondingly acidic solution into a quaternary compound in the composition. Cationic acylamidoamines are preferred according to the present invention.

Examples of amidoamines according to the present invention are: lauramidopropyl dimethylamine (Mackine 801), lauramidopropyl dimethylamine propionate, stearamidopropyl dimethylamine (Adogen® S18V or Tego® Amid S 18 or Incromine® SB), myristamidopropyl dimethylamine (Schercodine M), stearamidoethyl diethylamine (Lexamine 22), stearamidoethyl diethylamine phosphate, cocamidopropyl dimethylamine (Mackin® 101), ricinolamidopropyl dimethylamine (Mackine® 201), isostearamidopropyl dimethylamine (Mackine 401), oleamidopropyl dimethylamine (Mackine® 501), behenamidopropyl dimethylamine (Mackine 601, Incromine® BD), cocamidopropyl dimethylamine propionate (Mackalene® 117), cocamidopropyl dimethylamine lactate (Mackalene® 116), ricinoleamidopropyl dimethylamine lactate (Mackalene 216), stearamidopropyl dimethylamine lactate (Mackalene 316), behenamidopropyl dimethylamine lactate (Mackalene® 616), sunflowerseedamidopropyl dimethylamine lactate (Mackalene 1216), palmamidopropyl dimethylamine, palmamidopropyl dimethylamine lactate, palmamidopropyl dimethylamine propionate, oleamidopropyl dimethylamine glycolate, oleamidopropyl dimethylamine lactate; and examples of permanently cationic amidoamines are: Quaternium-33 (Swanol® Lanoquat DES-50), behenamidopropyl ethyldimonium ethosulfate (Schercoquat® BAS), behenamidopropyl PG-dimonium chloride (Lexquat® AMG-BEO), oleamidopropyl ethyldimonium ethosulfate, oleamidopropyl PG-dimonium chloride (Lexquat® AMG-O), cocamidopropyl ethyldimonium ethosulfate (Schercoquat® CAS), cocamidopropyltrimonium chloride (Empigen® CSC), ricinoleamidopropylethyldimonium ethosulfate, rinoleamidopropyltrimonium chloride, ricinoleamidopropyltrimonium methosulfate (Rewoquat® RTM 50), stearamidopropyl ethyldimonium ethosulfate (Schercoquat® SAS), stearamidopropyl trimonium methosulfate (Catagene® SA-70) or undecyleneamidopropyltrimonium methosulfate (Rewoquat® UTM 50), lauramidopropyl PG-dimonium chloride, canolamidopropyl ethyldimonium ethosulfate (Schercoquat® COAS).

Preferred amidoamines are lauramidopropyl dimethylamine, myristamidopropyl dimethylamine, stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine, palmamidopropyl dimethylamine, Quaternium-33, behenamidopropyl ethyldimonium ethosulfate, oleamidopropyl ethyldimonium etho sulfate, cocamidopropyltrimonium chloride, rinoleamidopropyltrimonium chloride, stearamidopropyl trimonium methosulfate.

The amidoamines stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine, palmamidopropyl dimethylamine, Quaternium-33, and behenamidopropyl ethyldimonium ethosulfate are particularly preferred.

Stearamidopropyl dimethylamine, cocamidopropyl dimethylamine, isostearamidopropyl dimethylamine, behenamidopropyl dimethylamine, and behenamidopropyl ethyldimonium ethosulfate are highly preferred.

Stearamidopropyl dimethylamine is most preferred.

The amidoamines recited above can be used individually or in any combinations with one another, being included in quantities of between 0.01 and 6.0 wt %, preferably in quantities from 0.01 to 3.0 wt %, and very particularly preferably in quantities from 10. to 2.5 wt %. The best results of all are obtained with quantities from 0.2 to 1.5 wt % based in each case on the total composition of the respective agent.

Ester oils are the third group of ingredients obligatorily necessary according to the present invention. “Ester oils” are to be understood as esters of C₆ to C₃₀ fatty acids with C₂ to C₃₀ fatty alcohols. The monoesters of fatty acids with alcohols having 2 to 24 carbon atoms are preferred. Examples of fatty-acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Examples of fatty-alcohol components in the ester oils are isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof. Isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred according to the present invention.

The ester oils can of course also be alkoxylated with ethylene oxide, propylene oxide, or mixtures of ethylene oxide and propylene oxide. The alkoxylation can be located both on the fatty-alcohol part and on the fatty-acid part, and also on both parts, of the ester oils. It is preferred according to the present invention, however, if the fatty alcohol was first alkoxylated and then was esterified with fatty acid. Formula (D4-II) depicts these compounds in generalized fashion.

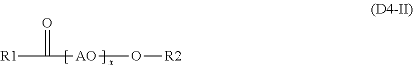

R1 here denotes a saturated or unsaturated, branched or unbranched, cyclic saturated or cyclic unsaturated acyl residue having 6 to 30 carbon atoms,

AO denotes ethylene oxide, propylene oxide, or butylene oxide,
X denotes a number between 1 and 200, preferably 1 and 100, particularly preferably between 1 and 50, very particularly preferably between 1 and 20, highly preferably between 1 and 10, and most preferably between 1 and 5,
R2 denotes a saturated or unsaturated, branched or unbranched, cyclic saturated or cyclic unsaturated alkyl, alkenyl, alkinyl, phenyl, or benzyl residue having 6 to 30 carbon atoms. Examples of fatty-acid components used as residue R1 in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Examples of the fatty-alcohol components as residue R2 in the ester oils are benzyl alcohol, isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof. An ester oil that is particularly preferred according to the present invention is obtainable, for example, under the INCI name PPG-3 Benzyl Ether Myristate.

Also to be understood as ester oils are:
dicarboxylic acid esters such as di-n-butyl adipate, di-(2-ethylhexyl) adipate, di-(2-ethylhexyl) succinate, and diisotridecyl acelaate, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate, as well as
symmetrical, asymmetrical, or cyclic esters of carbonic acid with fatty alcohols, for example glycerol carbonate or dicaprylyl carbonate (Cetiol® CC),
fatty acid partial glycerides, i.e. monoglycerides, diglycerides, and industrial mixtures thereof. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Oleic acid monoglycerides are preferably used.

It is of course also possible according to the present invention to use several ester oils simultaneously. Preferred ester oils are isopropyl myristate, glycerol carbonate, dicaprylyl carbonate, isopropyl palmitate, PPG-3 benzyl ether myristate, cetyl oleate, and oleyl erucate, as well as mixtures of at least two of these ester oils. Mixtures of ester oils are most preferred when one of the ester oils is dicaprylyl carbonate or isopropyl myristate. Mixtures of and having these two ester oils are highly preferred. When a mixture of ester oils having the two last-named oils is used, it is most preferred if isopropyl myristate and caprylyl carbonate are present at a ratio from 10:1 to 1:1, preferably 5:1 to 1:1, even more preferably 5:1 to 3:1, referring to the respective quantities of the ester oils.

Ester oils are used in the agents according to the present invention in a quantity from 0.01 to 7.5 wt %, preferably 0.01 to 5.0 wt %, particularly preferably 0.05 to 5.0 wt %, highly preferably from 0.2 to 5.0 wt %.

Cationic polymers are used as a fourth obligatory component of the compositions according to the present invention. Cationic polymers are selected from the following cationic polymers:

Suitable cationic polymers that are derived from natural polymers are cationic derivatives of polysaccharides, for example cationic derivatives of cellulose, starch, or guar. Cationic polysaccharides have the general formula G-O—B—N+RₐR_{b}R_{c} A⁻

G is an anhydroglucose residue, for example starch anhydroglucose or cellulose anhydroglucose;
B is a divalent connecting group, for example alkylene, oxyalkylene, polyoxyalkylene, or hydroxyalkylene;
Rₐ, R_{b} and R_{c} are mutually independently alkyl, aryl, alkylaryl, arylalkyl, alkoxyalkyl, or alkoxyaryl each having up to 18 carbon atoms, the total number of carbon atoms in Rₐ, R_{b}, and R_{c} preferably being a maximum of 20;
A⁻ is a usual counter anion and is preferably chloride.

Among the polymers derived from natural cationic polymers, cationic celluloses are preferred. Cationic celluloses having different degrees of substitution, cationic charge densities, nitrogen contents, and molecular weights are obtainable on the market. The cationic celluloses below in particular are highly preferred according to the present invention.

For example, Polyquaternium-67 is offered commercially under the names Polymer® SL or Polymer® SK (Amerchol). A further highly preferred cellulose is offered by the Croda company under the commercial name Mirustyle® CP. This is a trimonium and cocodimonium hydroxyethyl cellulose, constituting a derivatized cellulose, having the INCI-name Polyquaternium-72. Polyquaternium-72 can be used both in solid form and already predisssolved in aqueous solution.

Suitable cationic polymers that are derived from synthetic polymers are, for example, copolymers of 0.1 to 50% (based on the total number of monomers in the copolymer) monomers of foimula (Ia)

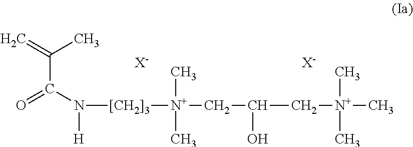

in which
X denotes chloride, sulfate, methosulfate,
monomers from the group of acrylamide, acrylic acid, methacrylic acid, and alkali-metal and ammonium salts of said acids, wherein the monomers constitute 50 to 99.9%, preferably 50 to 90% (based on the total number of monomers in the copolymer) of the copolymer. Particularly preferred monomers are acrylic acid or salts thereof (also mixed, i.e. partly neutralized acrylic acids), as well as acrylamide.

Regardless of which copolymers A are used in the agents according to the present invention, hair treatment agents according to the present invention in which copolymer A has a molar mass from 10,000 to 20 million gmol⁻¹, preferably from 100,000 to 10 million gmol⁻¹, more preferably from 500,000 to 5 million gmol⁻¹, and in particular from 1.1 million to 2.2 million gmol⁻¹, are preferred.

A highly preferred polymer that is constructed as depicted above is obtainable commercially under the name Polyquatemium-74.

A further highly preferred cationic synthetic polymer is poly(methacryloyloxyethyltrimethylammonium) chloride, a homopolymer (crosslinked, if desired) having the INCI name Polyquaternium-37. Such products are available commercially, for example, under the designations Rheocare® CTH (Cosmetic Rheologies) and Synthalen® CR (3V Sigma).

The homopolymer is used preferably in the form of a nonaqueous polymer dispersion. Polymer dispersions of this kind are obtainable commercially under the names Salcare® SC 95 and Salcare® SC 96.

Hair treatment agents according to the present invention contain, based on their weight, Polyquaternium-37 in quantities from 0.001 to 5 wt %, preferably 0.0025 to 2.5 wt %, particularly preferably 0.005 to 1 wt %, more preferably 0.0075 to 0.75 wt %, and in particular 0.01 to 0.5 wt %.

Amodimethicones are aminofunctional silicones. These have already been known for some time. Amodimethicones having morpholino groups in the molecule have, however, only recently become available. They optimize the properties of the composition according to the present invention in outstanding fashion. At least one 4-morpholinomethyl-substituted silicone of formula (V) is therefore very particularly preferably used as an aminofunctional silicone:

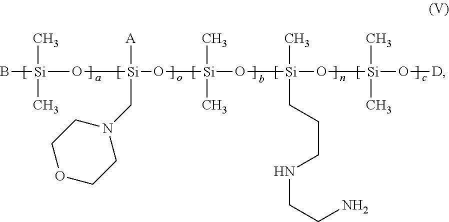

in which
A denotes a structural unit (I), (II), or (III) bound via —O—

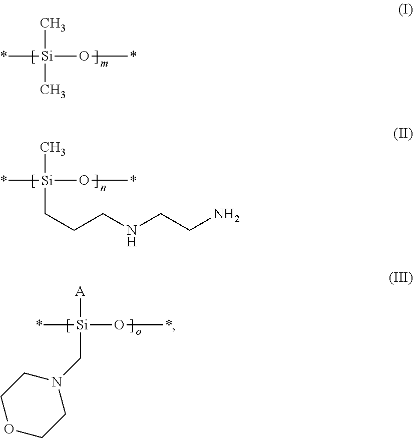

or an oligomeric or polymeric residue, bound via —O—, containing structural units of formulas (I), (II), or (III), or half of a connecting oxygen atom to a structural unit (III), or denotes —OH,
* denotes a bond to one of the structural units (I), (II), or (III), or denotes a terminal group B (Si-bound) or D (O-bound),
B denotes an —OH, —O—Si(CH₃)₃, —O—Si(CH₃)₂OH, —O—Si(CH₃)₂OCH₃ group,
D denotes an —H, —Si(CH₃)₃,—Si(CH₃)₂OH, —Si(CH₃)₂OCH₃ group,
a, b, and c denote integers between 0 and 1000, with the provision that a+b+c>0,
m, n, and o denote integers between 1 and 1000.

Aminofunctional silicones of this kind bear the INCI name Amodimethicone/Morpholinomethyl Silsesquioxane Copolymer. A particularly suitable amodimethicone is the product having the commercial name Wacker Belsil® ADM 8301E.

It can be particularly advantageous according to the present invention if exclusively the silicones recited above are used as silicones. The aforementioned cationic aminofunctional silicones are included in the compositions according to the present invention in quantities from 0.01 to 10 wt %, preferably in quantities from 0.05 to 7.5 wt %, and very particularly preferably in quantities from 0.1 to 7.5 wt %. The best results of all are obtained with quantities from 0.1 to 5 wt %, based in each case on the total composition of the respective agent.

The last obligatory component of the composition according to the present invention is sugar surfactants. It is highly preferred to use, as nonionic surfactants, those based on sugar. These are on the one hand preferably an alkyl- or alkenyloligoglucoside. These nonionic emulsifier agents represent known nonionic surfactants according to formula (I),

R¹O-[G]ₚ (I)

in which R¹ denotes an alkyl or alkenyl residue having 4 to 22 carbon atoms, G a sugar residue having 5 or 6 carbon atoms, and p denotes numbers from 1 to 10. Alkyl- and alkenyloligoglycosides can be derived from aldoses or ketoses having 5 or 6 carbon atoms, preferably from glucose. The preferred alkyl- and/or alkenyloligoglycosides are thus alkyl- and/or alkenyloligoglucosides. The index number p in the general formula (I) indicates the degree of oligomerization (DP), i.e. the distribution of mono- and oligoglycosides, and denotes a number between 1 and 10. Whereas p in the individual molecule must always be an integer, and here can assume especially the values p=1 to 6, the value p for a specific alkyloligoglycoside is an analytically ascertained calculated value that usually represents a fractional number. Alkyl- and/or alkenyloligoglycosides having an average degree of oligomerization p from 1.1 to 3.0 are preferably used. Those alkyl- and/or alkenyloligoglycosides whose degree of oligomerization is less than 1.7 and in particular between 1.2 and 1.4 are preferred. The alkyl or alkenyl residue R¹ can be derived from primary alcohols having 4 to 22, preferably 8 to 22 carbon atoms. Typical examples are butanol, capronyl alcohol, capryl alcohol, caprinyl alcohol, undecyl alcohol, lauryl alcohol, myristyl alcohol, cetyl alcohol, palmoleyl alcohol, stearyl alcohol, isostearyl alcohol, oleyl alcohol, elaidyl alcohol, petroselinyl alcohol, arachidyl alcohol, gadoleyl alcohol, behenyl alcohol, erucyl alcohol, brassidyl alcohol, natural fatty alcohols such as coconut alcohol, as well as industrial mixtures. Examples of products available commercially are the Oramix® grades of the Seppic company, for example Oramix® NS 10, Plantacare® grades, for example, Plantacare® 2000 UP, Plantacare® 1200 UP, Plantacare® 810 UP, Plantacare® 818 UP.

The sugar-based emulsifier agent can furthermore be a fatty acid N-alkylpolyhydroxyalkylamide of formula (II)

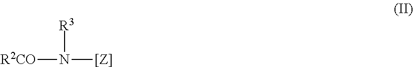

in which R²CO denotes an aliphatic acyl residue having 6 to 22 carbon atoms, R³ denotes hydrogen, an alkyl or hydroxyalkyl residue having 1 to 4 carbon atoms, and [Z] denotes a linear or branched polyhydroxylalkyl residue having 3 to 12 carbon atoms and 3 to 10 hydroxyl groups.

The fatty acid N-alkylpolyhydroxyalkylamides are preferably derived from reducing sugars having 5 or 6 carbon atoms, in particular from glucose. The preferred fatty acid N-alkylpolyhydroxyalkylamides therefore represent fatty acid N-alkylglucamides such as those reproduced by formula (III):

R²CO—NR³—CH₂—(CHOH)₄—CH₂OH (III).

It is preferable to use, as fatty acid N-alkylpolyhydroxyalkylamides, glucamides of formula (III) in which R³ denotes hydrogen or an alkyl group and R²CO denotes the acyl residue of hexanoic acid, octanoic acid, decanoic acid, lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid, or erucic acid, or industrial mixtures thereof. The polyhydroxyalkylamides can furthermore also be derived from maltose and palatinose.

The sugar-based nonionic surfactant is included in the agents used according to the present invention preferably in quantities from 0.001 to 3.0 wt %, based on the total agent. Quantities from 0.01 to 2.0 wt % are particularly preferred.

The compositions according to the present invention can of course also optionally contain, in addition to the essential ingredients described above, further usual ingredients. These are added in particular in order to impart further desirable properties, such as an action against dandruff, to the compositions, or to improve volume, etc. These ingredients will now be described.

Fatty alcohols (Fatal) that can be used are saturated, mono- or polyunsaturated, branched or unbranched fatty alcohols. Saturated and unbranched fatty alcohols preferably having a carbon chain length from C₆ to C₁₈, preferably C₈ to C₁₈, and very particularly preferably C₁₀ to C₁₆ are used. Mono- or polyunsaturated fatty alcohols as well as branched and unsaturated or branched and saturated fatty alcohols are used by preference with a carbon chain length from C₆ to C₃₀, preferably C₁₀ to C₂₂, and very particularly preferably from C₁₂ to C₂₂. Usable for purposes of the invention are, for example, decanol, octanol, octenol, dodecenol, decenol, octadienol, dodecadienol, decadienol, oleyl alcohol, erucyl alcohol, ricinol alcohol, lauryl alcohol, myristyl alcohol, capryl alcohol, caprinyl alcohol, linoleyl alcohol, linolenyl alcohol, this listing being intended to be exemplary and not limiting in nature. Also usable according to the present invention are those fatty alcohol cuts which represent a mixture of different fatty alcohols. Such substances are, for example, available for purchase under the designations Stenol® or Lanette® or Nafol® or Lorol®, e.g. Lorol® C8, Lorol® C14, Lorol® C18, Lorol® C8-18, HD-Ocenol®, Crodacol®, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16, or Isocarb® 24. It is of course also possible according to the present invention to use wool-wax alcohols such as those available for purchase under the designations Corona®, White Swan®, Coronet®, or Fluilan®. The softening point of the fatty alcohols usable according to the present invention is at most 45° C., preferably 15 to 40° C., particularly preferably 15 to 35° C., and highly preferably 15 to 28° C.

Fatty alcohols are used in quantities from 0.1 to 30 wt % based on the total preparation, preferably in quantities from 0.1 to 20 wt %.

The quantity of fatty alcohols used is 0.1 to 15 wt % based on the total agent. The quantity is particularly preferably 0.1 to 10 wt %; quantities from 0.1 to 5 wt % can be very particularly advantageous.

Fatty acids (Fatac) that can be used are linear and/or branched, saturated and/or unsaturated fatty acids having 6 to 30 carbon atoms. Fatty acids having 10 to 22 carbon atoms are preferred. Among those that may be recited are, for example, isostearic acids, such as the commercial products Emersol® 871 and Emersol® 875, and isopalmitic acids such as the commercial product Edenor® IP 95, as well as all other fatty acids marketed under the Edenor® commercial designations (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. The fatty acid cuts that are obtainable from coconut oil or palm oil are usually particularly preferred; the use of stearic acid is, as a rule, particularly preferred.

The quantity used is 0.1 to 15 wt % based on the total agent. The quantity is preferably 0.5 to 10 wt %, and quantities from 1 to 5 wt % can be very particularly advantageous.

Cosmetic oils can furthermore be used in addition to the active-agent combination (A) according to the present invention. These oily substances preferably have a melting point lower than 50° C., particularly preferably lower than 45° C., very particularly preferably lower than 40° C., highly preferably lower than 35° C., and most preferably the cosmetic oils are flowable at a temperature lower than 30° C. These oils will be more specifically defined and described below.

Included among the natural and synthetic cosmetic oils are, for example:
Vegetable oils. Examples of such oils are sunflower oil, olive oil, soy oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils.
Liquid paraffin oils, isoparaffin oils, and synthetic hydrocarbons, as well as di-n-alkyl ethers having a total of between 12 and 36 carbon atoms, in particular 12 to 24 carbon atoms, for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert-butyl ether, diisopentyl ether, di-3-ethyldecyl ether, tert-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, can be preferred.

Suitable natural oils are, for example, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn pulp oil, sea buckthorn seed oil, sesame oil, shea butter, soy oil, sunflower oil, grapeseed oil, walnut oil, or wild rose oil.

Natural or synthetic waxes (Fatwax) that can be used according to the present invention are solid paraffins or isoparaffins, carnauba waxes, beeswaxes, candelilla waxes, ozocerites, ceresin, spermaceti, sunflower wax, fruit waxes such as apple wax or citrus wax, microcrystalline waxes made from PE or PP. Such waxes are obtainable, for example, via Kahl & Co., Trittau.

The quantity used is 0.1 to 50 wt % based on the total agent, preferably 0.1 to 20 wt %, and particularly preferably 0.1 to 15 wt %, based on the total agent.

The following cationic surfactants in accordance with formula (Tkat-2) can also be used:

RCO—X—N⁺R¹R²R³A⁻ (Tkat-2)

R therein denotes a substituted or unsubstituted, branched or straight-chain alkyl or alkenyl residue having 11 to 35 carbon atoms in the chain,
X denotes —O—,
R¹ denotes an alkylene group, having 2 to 6 carbon atoms, which can be substituted or unsubstituted; in the event of a substitution, substitution with an —OH or —NH group is preferred,
R², R³ denote, mutually independently in each case, an alkyl or hydroxyalkyl group having 1 to 6 carbon atoms in the chain, wherein the chain can be straight or branched.
R5 denotes hydrogen or a C1 to C6 straight-chain or branched alkyl or alkenyl residue, which can also be substituted with a hydroxy group.

An example of such commercial products is Akypoquat® 131.

Esterquats in accordance with formula (Tkat1-2) can furthermore be used:

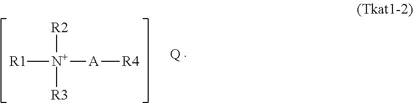

Residues R1, R2, and R3 therein are each mutually independent and can be identical or different. Residues R1, R2, and R3 signify:
a branched or unbranched alkyl residue having 1 to 4 carbon atoms, which can include at least one hydroxyl group, or
a saturated or unsaturated, branched or unbranched, or cyclic saturated or unsaturated alkyl residue having 6 to 30 carbon atoms, which can include at least one hydroxyl group, or
an aryl or alkaryl residue, for example phenyl or benzyl,
the residue (-A-R4), provided that at most two of the residues R1, R2, or R3 can denote this residue.

The residue —(X—R4) is included at least 1 to 3 times. In this, X denotes:
1) —(CH₂)n-, where n=1 to 20, preferably n=1 to 10, and particularly preferably n=1 to 5, or
   2) —(CH₂—CHR5-O)ₙ—, where n=1 to 200, preferably 1 to 100, particularly preferably 1 to 50, and particularly preferably 1 to 20, R5 having the meaning of hydrogen, methyl, or ethyl,
      and R4 denotes:
1) R6-O—CO—, in which R6 is a saturated or unsaturated, branched or unbranched, or a cyclic saturated or unsaturated alkyl residue having 6 to 30 carbon atoms, which can include at least one hydroxy group and which optionally can further be oxyethylated with 1 to 100 ethylene oxide units and/or 1 to 100 propylene oxide units, or
2) R7-CO—, in which R7 is a saturated or unsaturated, branched or unbranched, or a cyclic saturated or unsaturated alkyl residue having 6 to 30 carbon atoms, which can include at least one hydroxy group and which optionally can further be oxyethylated with 1 to 100 ethylene oxide units and/or 1 to 100 propylene oxide units, and Q denotes a physiologically acceptable organic or inorganic anion.

Such products are marketed, for example, under the trademarks Rewoquat®, Stepantex®, Dehyquart®, and Armocare®. The products Armocare® VGH-70—an N,N-bis(2-palmitoyloxyethyl)dimethylammonium chloride—as well as Dehyquart® F-75, Dehyquart® C-4046, Dehyquart® L80, Dehyquart® F-30, Dehyquart® AU-35, Rewoquat® WE18, Rewoquat® WE38 DPG, and Stepantex® VS 90 are examples of these esterquats.

Further compounds of formula (Tkat1-2) that are particularly preferred according to the present invention conform to the formula (Tkat1-2.1), the cationic betaine esters:

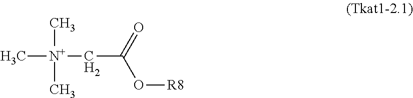

in which R8 is a saturated or unsaturated, branched or unbranched, or cyclic saturated or unsaturated alkyl residue having 6 to 30 carbon atoms, which can include at least one hydroxy group and which optionally can moreover be ethoxylated with 1 to 100 ethylene oxide units and/or 1 to 100 propylene oxide units.

The anion of all cationic compounds is selected from physiologically acceptable anions. Examples thereof that may be recited are the halide ions fluoride, chloride, bromide, iodide, sulfates of the general formula RSO₃⁻ in which R has the meaning of a saturated or unsaturated alkyl residue having 1 to 4 carbon atoms, or anionic residues of organic acids such as maleate, fumarate, oxalate, tartrate, citrate, lactate, or acetate.

The cationic surfactants recited above can be used individually or in any combinations with one another, included in quantities between 0.01 to 20 wt %, preferably in quantities from 0.01 to 10 wt %, and very particularly preferably in quantities from 0.1 to 7.5 wt %. The best results of all are obtained with quantities from 0.1 to 5 wt %, based in each case on the total composition of the respective agent.

Emulsifier agents usable according to the present invention are, for example:
addition products of 4 to 30 mol ethylene oxide and/or 0 to 5 mol propylene oxide with linear fatty alcohols having 8 to 22 carbon atoms, with fatty acids having 12 to 22 carbon atoms, and with alkylphenols having 8 to 15 carbon atoms in the alkyl group,
C12 to C22 fatty acid mono- and diesters of addition products of 1 to 30 mol ethylene oxide with polyols having 3 to 6 carbon atoms, in particular with glycerol,
addition products of ethylene oxide and polyglycerol with methylglucoside fatty acid esters, fatty acid alkanolamides, addition products of 5 to 60 mol ethylene oxide with castor oil and hardened castor oil,
partial esters of polyols having 3 to 6 carbon atoms with saturated fatty acids having 8 to 22 carbon atoms,
sterols, both from animal tissue (zoosterols, cholesterol, lanosterol) and from vegetable fats (phytosterols, ergosterol, stigmasterol, sitosterol), or from fungi and yeasts (mycosterols),
phospholipids (lecithins, phosphatidylcholines),
fatty acid esters of sugars and sugar alcohols, such as sorbitol,
polyglycerols and polyglycerol derivatives, for example polyglycerol poly-12-hydroxystearate (commercial product Dehymuls® PGPH).

Further emulsifier agents for the compositions according to the present invention can be phospholipids. Both natural and synthetic phospholipids are usable as a phospholipid. Phospholipids of natural origin are preferred. Compounds of formula (Phosphol-I) are usable in particular as phospholipids:

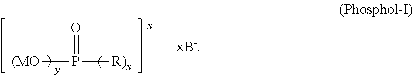

In formula (Phosphol-I), y denotes an integer from 0 to 2 and x an integer from 1 to 3, with the provision that the sum of x and y equals 3.

In phospholipids of formula (Phosphol-I) M furthermore denotes hydrogen, an equivalent of an alkali-metal or alkaline-earth-metal cation, an ammonium cation, or an alkyl residue having 1 to 4 carbon atoms that is optionally substituted with one or more hydroxy group(s).

Compounds in which M denotes a sodium cation are particularly preferred.

In addition, B in formula (Phosphol-I) for the phospholipids to be used according to the present invention denotes an equivalent of a physiologically acceptable anion. Suitable anions are, for example, chloride, bromide, iodide, sulfate, perchlorate, tetrafluoroborate, tetraphenylborate, and tetrachlorozincate. The chloride ion is preferred.

R in formula (Phosphol-I) denotes a residue of formula (II)

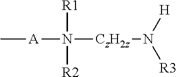

in which z denotes an integer from 1 to 4, in particular 3, and R¹ and R² mutually independently denote a C₁ to C₄ alkyl residue that is optionally substituted with one or more hydroxy group(s) or with an acyl group.

According to the present invention, A denotes one of the units —O—CH₂—CH₂—CH₂—, —O—CH₂—CH₂—, or —O—CH₂—CHOH—CH₂—, the —O—CH₂—CHOH—CH₂— unit being particularly preferred.

The residue R³ denotes
(a) a branched or unbranched saturated C₈ to C₁₈ acyl residue, or
(b) a branched or unbranched mono- or polyunsaturated C₈ to C₁₈ acyl residue.

Particularly preferred saturated residues R³ are the stearic acid residue and the residues of the mixture of fatty acid obtained from coconut oil.

The linoleic acid residue is a particularly preferred unsaturated residue R³.

Examples of the C₁ to C₄ alkyl groups recited as substituents in the compounds according to the present invention are methyl, ethyl, propyl, isopropyl, and butyl groups. Methyl groups are preferred alkyl groups. Methyl groups are very particularly preferred.

Very particularly preferred phospholipids of formula (Phosphol-I) are the substances known by the INCI names Linoleamidopropyl PG-Dimonium Chloride Phosphate, Cocamidopropyl PG-Dimonium Chloride Phosphate, and Stearamidopropyl PG-Dimonium Chloride Phosphate. These are marketed, for example, by the Mona company under the commercial names Phospholipid EFA®, Phospholipid PTC®, and Phospholipid SV®.

Glycerophospholipids, which are obtained e.g. as lecithins or phosphatidylcholines, for example, from egg yolk or plant seeds, in particular soybeans, are also used as phospholipids according to the present invention. Phospholipids are, in particular, phosphoglycerides.

Glycerophospholipids particularly suitable according to the present invention are obtained from soybeans. Particularly preferred thereamong are phosphatidylcholines, phosphatidylethanolamines, phosphatidylserines, and phosphatidylinositols, as well as mixtures of these substances.

Particularly preferred phosphatidylcholines have the formula (Phosphol-II)

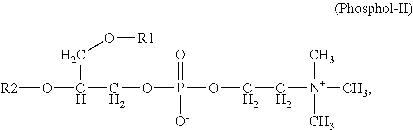

in which the residues R¹ and R², mutually independently in each case, represent an acyl group made of fatty acids having a carbon number from 8 to 30 carbon atoms, preferably 10 to 24, and particularly preferably 12 to 22 carbon atoms. The fatty acid residues can be both saturated and mono- or polyunsaturated. The saturated acyl residues of C₁₂ to C₂₂ fatty acids are preferred. The acyl residues of myristic acid, palmitic acid, stearic acid, arachidic acid, and behenic acid are particularly preferred.

Phosphatidylethanolamines preferred according to the present invention are those of formula (Phosphol-IIa) or (Phosphol-IIb)

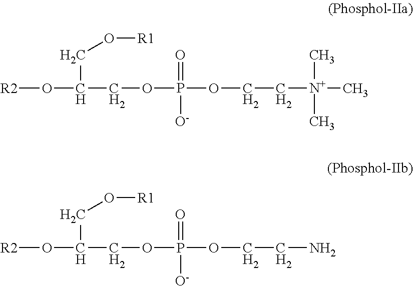

in which the residues R¹ and R² have the same meanings as presented for formula (Phosphol-II).

Phosphatidylethanolamines in which R¹ and R² mutually independently represent saturated acyl residues of fatty acids having 16 or 18 carbon atoms, in particular a palmitoyl or stearoyl residue, are particularly preferred.

Phosphatidylserines preferred according to the present invention are those having the structural formula (IIIa) or (IIIb)

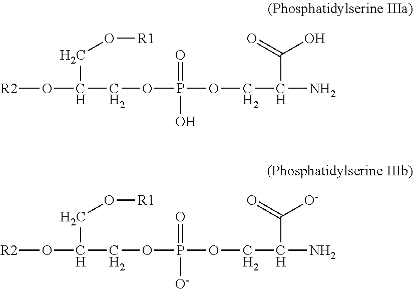

in which R¹ and R² have the same meanings as presented for formula (Phosphol-II). Phosphatidylserines in which R¹ and R² mutually independently represent saturated acyl residues of fatty acids having 16 or 18 carbon atoms, in particular a palmitoyl or stearoyl residue, are particularly preferred.

Phosphatidylinositols preferred according to the present invention have the structural formula (IVa) or (IVb)

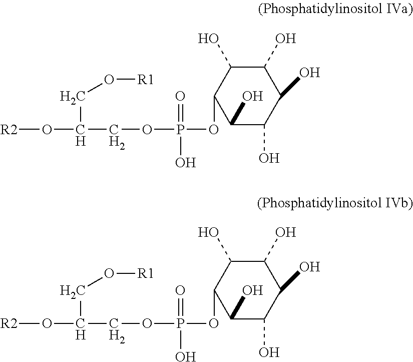

in which R¹ and R² have the same meanings as indicated for formula (Phosphol-II). Acyl residues of palmitic acid, stearic acid, and arachidic acid are preferred for R¹; a stearic acid acyl residue is particularly preferred. R² particularly preferably represents a linear saturated C₂₀ fatty acid acyl residue (arachoyl residue).

Glycerophospholipids used according to the present invention have an iodine number of at most 10, preferably at most 5.

It is also possible according to the present invention to use a mixture of several phospholipids.

A phospholipid preferred according to the present invention is available commercially under the name Emulmetik® 100 (Cognis). Phospholipids according to the present invention are included in the agents in concentrations from 0.1 wt % up to 7.5 wt %, preferably from 0.1 wt % up to 5 wt %, very particularly preferably in quantities from 0.1 wt % up to 3 wt %, and highly preferably in quantities from 0.1 up to 1.5 wt %.

Agents according to the present invention include emulsifier agents preferably in quantities from 0.1 to 25 wt %, in particular 0.5 to 15 wt %, based on the total agent.

Cationic polymers can be homo- or copolymers, the quaternary nitrogen groups being included either in the polymer chain or preferably as a substituent on one or more of the monomers. Ammonium-group-containing monomers can be copolymerized with non-cationic monomers. Suitable cationic monomers are unsaturated, radically polymerizable compounds that carry at least one cationic group, in particular ammonium-substituted vinyl monomers, for example trialkylmethacryloxyalkylammonium, trialkylacryloxyalkylammonium, dialkyl diallylammonium, and quaternary vinylammonium monomers having cyclic groups that include cationic nitrogens, such as pyridinium, imidazolium, or quaternary pyrrolidones, e.g. alkylvinylimidazolium, alkylvinylpyridinium, or alkyvinylpyrrolidone salts. The alkyl groups of these monomers are preferably lower alkyl groups, for example C1 to C7 alkyl groups, particularly preferably C1 to C3 alkyl groups.

Ammonium-group-containing monomers can be copolymerized with non-cationic monomers. Suitable comonomers are, for example, acrylamide, methacrylamide; alkyl and dialkyl acrylamide, alkyl and dialkyl methacrylamide, alkyl acrylate, alkyl methacrylate, vinylcaprolactone, vinylcaprolactam, vinylpyrrolidone, vinyl esters, e.g. vinyl acetate, vinyl alcohol, propylene glycol, or ethylene glycol, wherein the alkyl groups of these monomers are preferably C1 to C7 alkyl groups, particularly preferably C1 to C3 alkyl groups.

Suitable polymers having quaternary amine groups are, for example, the polymers described in the CTFA Cosmetic Ingredient Dictionary under the “Polyquaternium” designations, such as methylvinylimidazolium chloride/vinylpyrrolidone copolymer (Polyquatemium-16), or quaternized vinylpyrrolidone/dimethylaminoethyl methacrylate copolymer (Polyquaternium-11).

Suitable among the cationic polymers that can be included in the agent according to the present invention is, for example, the vinylpyrrolidone/dimethylaminoethyl methacrylate methosulfate copolymer that is marketed under the commercial names Gafquat® 755 N and Gafquat® 734 by the GAF company, USA, and of which Gafquat® 734 is particularly suitable. Further cationic polymers are, for example, the copolymer of polyvinylpyrrolidone and imidazolimine methochloride marketed by BASF, Germany, under the trade name Luviquat® HM 550, the terpolymer of dimethyldiallylammonium chloride, sodium acrylate, and acrylamide marketed by the Calgon company, USA, under the trade name Merquat® Plus 3300, and the vinylpyrrolidone/methacrylamidopropyltrimethylammonium chloride copolymer marketed by the ISP company under the trade name Gafquat® HS 100.

Homopolymers of the general formula (P1):

—{CH₂—[CR¹COO—(CH₂)ₘN⁺R²R³R⁴]}ₙX⁻,

in which R¹═—H or —CH₃, R², R³, and R⁴ are selected mutually independently from C1 to 4 alkyl, alkenyl, or hydroxyalkyl groups, m=1, 2, 3, or 4, n is a natural number, and
X⁻ is a physiologically acceptable organic or inorganic anion,
are preferred. In the context of these polymers, those for which at least one of the following conditions applies:

R¹ denotes a methyl group, R², R³, and R⁴ denote methyl groups, m has a value of 2 are preferred according to the present invention.

Halide ions, sulfate ions, phosphate ions, methosulfate ions, as well as organic ions such as lactate, citrate, tartrate, and acetate ions are appropriate, for example, as physiologically acceptable counter ions X⁻. Halide ions, in particular chloride, are preferred.

Suitable cationic guar derivatives are marketed under the commercial designation Jaguar® and have the INCI name Guar Hydroxypropyltrimonium Chloride. Particularly suitable cationic guar derivatives are additionally available commercially from the Hercules company under the designation N-Hance®. Further cationic guar derivatives are marketed by the Cognis company under the designation Cosmedia®. A preferred cationic guar derivative is the commercial product AquaCat® of the Hercules company. This raw material is a cationic guar derivative that is already predissolved.

A suitable chitosan is marketed, for example, by the Kyowa Oil & Fat company, Japan, under the trade name Flonac®. A preferred chitosan salt is chitosonium pyrrolidonecarboxylate, which is marketed e.g. under the designation Kytamer® PC by the Amerchol company, USA. Further chitosan derivatives are readily available commercially under the commercial designations Hydagen® CMF, Hydagen® HCMF, and Chitolam® NB/101.

Further preferred cationic polymers are, for example:
cationized alkyl polyglycosides,
cationized honey, for example the commercial product Honeyquat® 50,
polymeric dimethyldiallylammonium salts and copolymers thereof with esters and amides of acrylic acid and methacrylic acid. The products obtainable commercially under the designations Merquat® 100 (poly(dimethyldiallylammonium) chloride) and Merquat® 550 (dimethyldiallylammonium chloride/acrylamide copolymer) are examples of such cationic polymers,
vinylpyrrolidone/vinylimidazolium methochloride copolymers, such as those offered under the designations Luviquat® FC 370, FC 550, and HM 552,
quaternized polyvinyl alcohol,
and the polymers known by the names Polyquaternium-2, Polyquaternium-17, Polyquaternium-18, and Polyquaternium-27, having quaternary nitrogen atoms in the main polymer chain,
vinylpyrrolidone/vinylcaprolactam/acrylate terpolymers such as those having acrylic acid esters and acrylic acid amides as a third monomer module, and offered commercially e.g. under the designation Aquaflex® SF 40.

Also usable according to the present invention are the copolymers of vinylpyrrolidone such as those obtainable as the commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Cationized protein hydrolysates are further to be included among the cationic polymers, wherein the underlying protein hydrolysate can derive from animals, for example from collagen, milk, or keratin, from plants, for example from wheat, corn, rice, potatoes, soy, or almonds, from marine life forms, for example from fish collagen or algae, or from biotechnologically obtained protein hydrolysates. Typical examples of cationic protein hydrolysates and derivatives according to the present invention that may be recited are the products that are recited under the INCI names in the “International Cosmetic Ingredient Dictionary and Handbook” (seventh edition 1997, The Cosmetic, Toiletry, and Fragrance Association, 1101 17th Street, N.W., Suite 300, Washington, DC 20036-4702), and are available commercially.

The additional cationic polymers are included in the compositions according to the present invention preferably in quantities from 0.01 to 10 wt %, based on the total agent. Quantities from 0.05 to 5 wt % are particularly preferred.

Amphoteric polymers can also be used as polymers. The term “amphoteric polymers” encompasses both those polymers which include in the molecule both free amino groups and free —COOH or —SO₃H groups and are capable of forming internal salts, and zwitterionic polymers, which include quaternary ammonium groups and —COO⁻ or —SO₃⁻ groups in the molecule, and those polymers which include —COOH or —SO₃H groups and quaternary ammonium groups.

Amphoteric and/or cationic polymers according to the present invention are those polymerizates in which a cationic group derives from at least one of the following monomers:
(i) monomers having quaternary ammonium groups of the general formula (Mono 1)

R¹—CH═CR²—CO—Z—(CₙH₂ₙ)—N⁽⁺⁾R³R⁴R⁵ (Mono1),
in which R¹ and R² mutually independently denote hydrogen or a methyl group and R³, R⁴, and R⁵ mutually independently denote alkyl groups having 1 to 4 carbon atoms, Z denotes an NH group or an oxygen atom, n is an integer from 2 to 5, and A⁽⁻⁾ is the anion of an organic or inorganic acid,
(ii) monomers having quaternary ammonium groups of the general formula (Mono2)

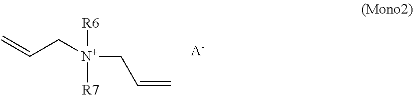

in which R⁶ and R⁷ mutually independently denote a (C₁ to C₄) alkyl group, in particular a methyl group, and
   A⁻ is the anion of an organic or inorganic acid,
(iii) monomeric carboxylic acids of the general formula (Mono3)

R⁸—CH═CR⁹—COOH (Mono3)
in which R⁸ and R⁹ mutually independently are hydrogen or methyl groups.

Those polymerizates in which the monomers used are of type (i) in which R³, R⁴, and R⁵ are methyl groups, Z is an NH group, and A⁽⁻⁾ is a halide, methoxysulfate, or ethoxysulfate ion, are particularly preferred; acrylamidopropyltrimethylammonium chloride is a particularly preferred monomer (i). Acrylic acid is preferably utilized as monomer (ii) for the aforesaid polymerizates.

Particularly preferred amphoteric polymers are copolymers of at least one monomer (Mono1) or (Mono2) with the monomer (Mono3), in particular copolymers of monomers (Mono2) and (Mono3). Amphoteric polymers used very particularly preferably according to the present invention are copolymerizates of diallyldimethylammonium chloride and acrylic acid. These copolymerizates are marketed under the INCI name Polyquaternium-22, inter alia with the commercial name Merquat® 280 (Nalco).

Furthermore, the amphoteric polymers according to the present invention can additionally contain, besides a monomer (Mono1) or (Mono2) and a monomer (Mono3), a monomer (Mono4)
(iv) monomeric carboxylic acid amides of the general formula (Mono4)

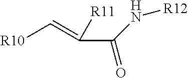

in which R¹⁰ and R¹¹ mutually independently are hydrogen or methyl groups, and R¹² denotes a hydrogen atom or a (C₁ to C₈) alkyl group.

Amphoteric polymers based on a comonomer (Mono4) that are used very particularly preferably according to the present invention are terpolymers of diallyldimethylammonium chloride, acrylamide, and acrylic acid. These copolymerizates are marketed under the INCI name Polyquaternium-39, inter alia with the commercial name Merquat® Plus 3330 (Nalco).

Amphoteric polymers can in general be used according to the present invention both directly and in a salt form that is obtained by neutralizing the polymerizates, for example using an alkali hydroxide.

Amphoteric polymers are included in the agents according to the present invention preferably in quantities from 0.01 to 10 wt % based on the total agent. Quantities from 0.01 to 5 w % are particularly preferred.

The anionic polymers are anionic polymers that comprise carboxylate groups and/or sulfonate groups. Examples of anionic monomers of which such polymers can be made are acrylic acid, methacrylic acid, crotonic acid, maleic acid anhydride, and 2-acrylamido-2-methylpropanesulfonic acid. The acid groups can be present entirely or partially as a sodium, potassium, ammonium, mono- or triethanolammonium salt.

Preferred monomers are 2-acrylamido-2-methylpropanesulfonic acid and acrylic acid.

Anionic polymers that include 2-acrylamido-2-methylpropanesulfonic acid as the only monomer or co-monomer have proven to be very particularly effective, wherein the sulfonic acid group can be present entirely or partly as a sodium, potassium, ammonium, mono- or triethanolammonium salt.

The homopolymer of 2-acrylamido-2-methylpropanesulfonic acid that is obtainable commercially, for example, under the designation Rheothik® 11-80 is particularly preferred.

Within this embodiment it can be preferred to use copolymers of at least one anionic monomer and at least one nonionogenic monomer. Reference is made to the substances listed above regarding the anionic monomers. Preferred nonionogenic monomers are acrylamide, methacrylamide, acrylic acid esters, methacrylic acid esters, vinylpyrrolidone, vinyl ethers, and vinyl esters.

Preferred anionic copolymers are acrylic acid/acrylamide copolymers and in particular polyacrylamide copolymers with sulfonic acid group-containing monomers. A polymer of this kind is included in the commercial product Sepigel® 305 of the SEPPIC Company.

The sodium acryloyl dimethyl taurate copolymers marketed, under the designation Simulgel® 600, as a compound with isohexadecane and polysorbate-80 have also proven particularly effective according to the present invention.

Similarly preferred anionic homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

Copolymers of maleic acid anhydride and methyl vinyl ether, in particular those having crosslinks, are also color-retaining polymers. A maleic acid/methyl vinyl ether copolymer crosslinked with 1,9-decadiene is obtainable commercially under the name Stabileze® QM.

Anionic polymers are included in the agents according to the present invention preferably in quantities from 0.05 to 10 wt % based on the total agents. Quantities from 0.1 to 5 wt % are particularly preferred.

A polyurethane that is very particularly preferred according to the present invention is on the market under the commercial designation Luviset® PUR (BASF).

In a further embodiment, the agents according to the present invention can include nonionogenic polymers.

Suitable nonionogenic polymers are, for example:
Vinylpyrrolidone/vinyl ester copolymers such as those marketed, for example, under the trademark Luviskol® (BASF). Luviskol® VA 64 and Luviskol® VA 73, which are each vinylpyrrolidone/vinyl acetate copolymers, are likewise preferred nonionic polymers.
Cellulose ethers such as hydroxypropyl cellulose, hydroxyethyl cellulose, and methyihydroxypropyl cellulose, such as those marketed, for example, under the trademarks Culminal® and Benecel® (AQUALON) and Natrosol® grades (Hercules).
Starch and derivatives thereof, in particular starch ethers, for example Structure® XL (National Starch), a multifunctional, salt-tolerant starch,
shellac,
polyvinylpyrrolidones such as those marketed, for example, under the name Luviskol® (BASF).

Nonionic polymers are included in the compositions according to the present invention preferably in quantities from 0.05 to 10 wt % based on the total agent. Quantities from 0.1 to 5 wt % are particularly preferred.

It is also possible according to the present invention for the preparations that are used to include multiple, in particular two different polymers of the same charge, and/or one ionic and one amphoteric and/or nonionic polymer.

Polymers (P) are included in the compositions used according to the present invention preferably in quantities from 0.01 to 30 wt % based on the total composition. Quantities from 0.01 to 25, in particular from 0.01 to 15 wt % are particularly preferred.

As a further optional ingredient, the compositions according to the present invention preferably include at least one silicone polymer selected from the group of dimethiconols and/or the group of aminofunctional silicones and/or the group of dimethicones and/or the group of cyclomethicones. These ingredients are described below.

Dimethicones according to the present invention can be both linear and branched, and also cyclic or cyclic and branched. Linear dimethicones can be represented by the following structural formula (Si1):

(SiR¹₃)—O—(SiR²₂—O—)ₓ—(SiR¹₃) (Si1).

Branched dimethicones can be represented by the structural formula (Si1.1):

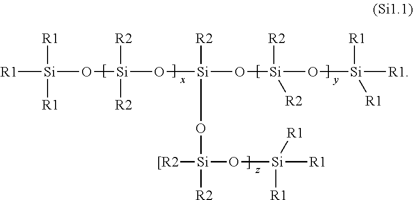

Residues R¹ and R² denote, mutually independently in each case, hydrogen, a methyl residue, a C2 to C30 linear, saturated or unsaturated hydrocarbon residue, a phenyl residue, and/or an aryl residue. The numbers x, y, and z are integers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscometer in accordance with Dow Corning Corporate Test Method CTM 0004 of Jul. 20, 1970. Preferred viscosities are between 1000 and 5,000,000 cPs; very particularly preferred viscosities are between 10,000 and 3,000,000 cPs. The most preferred range is between 50,000 and 2,000,000 cPs. Viscosities around the range of approximately 60,000 cPs are highly preferred. Reference may be made here, for example, to the product “Dow Corning 200, 60,000 cSt.”

Particularly preferred cosmetic or dermatological preparations according to the present invention are characterized in that they include at least one silicone of formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20, and in particular 0 to 10.

Dimethicones (Si1) are included in the compositions according to the present invention in quantities from 0.01 to 10 wt %, preferably 0.01 to 8 wt %, particularly preferably 0.1 to 7.5 wt %, and in particular 0.1 to 5 wt %, based on the total composition.

Particularly preferred agents according to the present invention include one or more aminofunctional silicones. Such silicones can be described, for example, by formula (Si-2)

M(RₐQ_{b}SiO_{(4-a-b)/2})ₓ(R_{c}SiO_{(4-c)/2})_{y}M (Si-2);

in the above formula,
R is a hydrocarbon or a hydrocarbon residue having 1 to approximately 6 carbon atoms,
Q is a polar residue of the general formula —R¹HZ, in which
   R¹ is a divalent connecting group that is bound to hydrogen and to the Z residue, assembled from carbon and hydrogen atoms, carbon, hydrogen, and oxygen atoms, or carbon, hydrogen, and nitrogen atoms, and
   Z is an organic aminofunctional residue that includes at least one aminofunctional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2,000, preferably from approximately 3 to approximately 50, and most preferably from approximately 3 to approximately 25, and
y is a number in the range from approximately 20 to approximately 10,000, preferably from approximately 125 to approximately 10,000, and most preferably from approximately 150 to approximately 1,000, and
M is a suitable silicone terminal group as known in the existing art, preferably trimethylsiloxy.

Z according to formula (Si-2) is an organic aminofunctional residue that includes at least one functional amino group. One possible formula for the aforesaid Z is NH(CH₂)_{z}NH₂, in which z is an integer greater than or equal to 1. Another possible formula for the aforesaid Z is —NH(CH₂)_{z}(CH₂)_{zz}NH, in which both z and zz mutually independently are an integer greater than or equal to 1, said structure encompassing diamino ring structures such as piperazinyl. The aforesaid Z is most preferably an —NHCH₂CH₂NH₂ residue. Another possible formula for the aforesaid Z is —N(CH₂)_{z}(CH₂)_{zz}NX₂ or —NX₂, in which each X is selected, independently of X₂, from the group consisting of hydrogen and alkyl groups having 1 to 12 carbon atoms, and zz is 0.

Q according to formula (Si-2) is most preferably a polar aminofunctional residue of the formula —CH₂CH₂CH₂NHCH₂CH₂NH₂.

In formula (Si-2), a assumes values in the range from 0 to 2, b assumes values in the range from 2 to 3, a+b is less than or equal to 3, and c is a number in the range from 1 to 3.

Cationic silicone oils, for example the commercially obtainable products Dow Corning (DC) 929 Emulsion, DC 2-2078, DC 5-7113, SM-2059 (General Electric), and SLM-55067 (Wacker) are suitable according to the present invention.

Particularly preferred agents according to the present invention are characterized in that they include at least one aminofunctional silicone of formula (Si3-a)

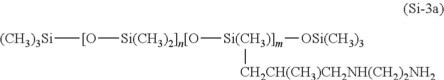

in which m and n are numbers whose sum (m+n) is between 1 and 2000, preferably between 50 and 150, wherein n assumes values preferably from 0 to 1999 and in particular from 49 to 149, and m preferably assumes values from 1 to 2000, in particular from 1 to 10.

These silicones are referred to according to the INCI declaration as Trimethylsilylamodimethicones and are obtainable, for example, under the designation Q2-7224 (manufacturer: Dow Corning; a stabilized trimethylsilylamodimethicone).

Also particularly preferred are agents according to the present invention that include at least one aminofunctional silicone of formula (Si-3b)

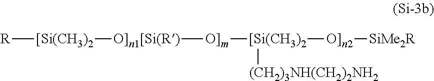

in which
R denotes —OH, an (optionally ethoxylated and/or propoxylated) (C₁ to C₂₀) alkoxy group, or a —CH₃ group,
R′ denotes —OH, a (C₁ to C₂₀) alkoxy group, or a —CH₃ group, and
m, n1, and n2 are numbers whose sum (m+n1+n2) is between 1 and 2000, preferably between 50 and 150, wherein the sum (n1+n2) assumes values preferably from 0 to 1999 and in particular from 49 to 149, and m preferably assumes values from 1 to 2000, in particular from 1 to 10.

These silicones are referred to according to the INCI declaration as Amodimethicones or as functionalized Amodimethicones, for example Bis(C13-15 Alkoxy) PG Amodimethicone (obtainable e.g. as a commercial product: DC 8500 of the Dow Corning company), Trideceth-9 PG-Amodimethicone (obtainable e.g. as a commercial product: Silcare Silicone SEA of the Clariant company).

Suitable diquaternary silicones are selected from compounds of the general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c),

wherein the residues R1 to R6 mutually independently signify C1 to C22 alkyl residues that can include hydroxy groups, and wherein preferably at least one of the residues comprises at least 8 carbon atoms and the remaining residues comprise 1 to 4 carbon atoms, the residues R7 to R12 mutually independently are identical or different and signify C1 to C10 alkyl or phenyl, A signifies a divalent organic connecting group,
n is a number from 0 to 200, preferably from 10 to 120, particularly preferably from 10 to 40,
and X⁻ is an anion.

The divalent connecting group is preferably a C1 to C12 alkylene or alkoxyalkylene group that can be substituted with one or more hydroxyl groups.

Particularly preferably, the group is —(CH₂)₃—O—CH₂—CH(OH)—CH₂—.

The anion X⁻ can be a halide ion, an acetate, an organic carboxylate, or a compound of the general formula RSO₃⁻ in which R has the meaning of C1 to C4 alkyl residues.

A preferred diquaternary silicone has the general formula (Si3d)

[RN⁺Me₂-A-(SiMe₂O)ₙ—SiMe₂-A-N⁺Me₂R]2CH₃COO⁻ (Si3d),

wherein A is the group —(CH₂)₃—O—CH₂—CH(OH)—CH₂—,
R is an alkyl residue having at least 8 carbon atoms, and n is a number from 10 to 120.

Suitable silicone polymers having two terminal quaternary ammonium groups are known by the INCI name Quaternium-80. These are dimethylsiloxanes having two terminal trialkylammonium groups. Diquaternary polydimethylsiloxanes of this kind are marketed by the Evonik company under the commercial names Abil® Quat 3270, 3272, and 3474.

Cosmetic or dermatological preparations preferred according to the present invention are characterized in that they contain, based on their weight, 0.01 to 10 wt %, preferably 0.01 to 8 wt %, particularly preferably 0.1 to 7.5 wt %, and in particular 0.2 to 5 wt % aminofunctional silicone(s) and/or diquatemary silicone.

The compositions according to the present invention can include at least one polyammonium-polysiloxane compound as a silicone. Polyammonium-polysiloxane compounds can be acquired, for example, from GE Bayer Silicones under the commercial name Baysilone®. The products having the designations Baysilone TP 3911, SME 253, and SFE 839 are preferred in this context. It is very particularly preferred to use Baysilone TP 3911 as an active component of the compositions according to the present invention. Polyammonium-polysiloxane compounds are used in the compositions according to the present invention in a quantity from 0.01 to 10 wt %, preferably 0.01 to 7.5, particularly preferably 0.01 to 5.0 wt %, very particularly preferably from 0.05 to 2.5 wt %, referring in each case to the total composition.

Further cationic aminofunctional silicone polymers can also be used. EP 1887024 A1 describes novel cationic aminofunctional silicones that in particular improve shine in agents for the care of surfaces, for example human hair. These cationic silicone polymers are notable for the fact that they comprise a silicone skeleton as well as at least one polyether part and furthermore at least one part having an ammonium structure. Examples of the preferred cationic silicone polymers for purposes of the present invention are, besides the compounds of the aforementioned EP 1887024 A1, furthermore in particular the compounds having the INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22, as well as Silicone Quaternium-2 Panthenol Succinate and Silicone Quaternium-16/Glycidyl Dimethicone Crosspolymer. Silicone Quaternium-22 is, in particular, most preferred. This raw material is marketed, for example, by the Evonik company under the commercial name Abil® T-Quat 60.

The aforementioned cationic aminofunctional silicone polymers are included in the compositions according to the present invention in quantities from 0.01 to 20 wt %, preferably in quantities from 0.05 to 10 wt %, and very particularly preferably in quantities from 0.1 to 7.5 wt %. The best results of all are obtained with quantities from 0.1 to 5 wt %, based in each case on the total composition of the respective agent. It can be particularly advantageous according to the present invention if exclusively the silicones recited above are used as silicones.

The cyclic dimethicones referred to according to INCI as Cyclomethicones are also usable with preference according to the present invention. Cosmetic or dermatological preparations according to the present invention that include at least one silicone of formula (Si-4)

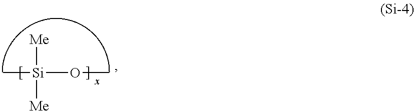

in which x denotes a number from 3 to 200, preferably from 3 to 10, more preferably from 3 to 7, and in particular 3, 4, 5, or 6, are preferred here.

Agents likewise preferred according to the present invention are characterized in that they include at least one silicone of formula (Si-5)

R₃Si-[O—SiR₂]ₓ—(CH₂)ₙ[O—SiR₂]_{y}—O—SiR₃ (Si-5),

in which R denotes identical or different residues from the group —H, phenyl, benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y respectively denote a number from 0 to 200, preferably from 0 to 10, more preferably from 0 to 7, and in particular 0, 1, 2, 3, 4, 5, or 6, and n denotes a number from 0 to 10, preferably from 1 to 8, and in particular 2, 3, 4, 5, 6.

Water-soluble silicones can be included in the compositions according to the present invention as further silicones besides dimethicones, dimethiconols, amodimethicones, and/or cyclomethicones.

Corresponding hydrophilic silicones are selected, for example, from the compounds of formulas (Si-6) and/or (Si-7). Particularly preferred silicone-based water-soluble surfactants are selected from the group of dimethicone polyols that are preferably alkoxylated, in particular polyethoxylated or polypropoxylated.

“Dimethicone polyols” are understood according to the present invention preferably as polyoxyalkylene-modified dimethylpolysiloxanes of the general formulas (Si-6) or (Si-7):

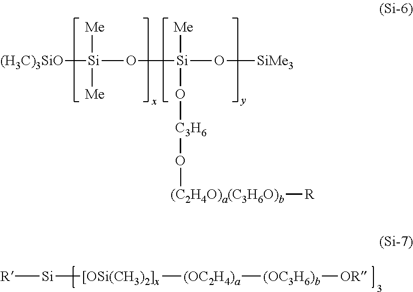

in which the residue R denotes a hydrogen atom, an alkyl group having 1 to 12 carbon atoms, an alkoxy group having 1 to 12 carbon atoms, or a hydroxyl group, the residues R′ and R″ signify alkyl groups having 1 to 12 carbon atoms, x denotes an integer from 1 to 100, preferably from 20 to 30, y denotes an integer from 1 to 20, preferably from 2 to 10, and a and b denote integers from 0 to 50, preferably from 10 to 30.

Particularly preferred dimethicone copolyols for purposes of the invention are, for example, the products marketed commercially under the trade name SILWET (Union Carbide Corporation) and DOW CORNING. Dimethicone copolyols particularly preferred according to the present invention are Dow Corning 190 and Dow Corning 193.

Dimethicone copolyols are in the compositions according to the present invention in quantities from 0.01 to 10 wt %, preferably 0.01 to 8 wt %, particularly preferably 0.1 to 7.5 wt %, and in particular 0.1 to 5 wt % dimethicone copolyol, based on the composition.

Lastly, dimethiconols (Si8) are understood as silicone compounds. Dimethiconols according to the present invention can be both linear and branched, as well as cyclic or cyclic and branched. Linear dimethiconols can be represented by the following structural formula (Si8-I):

(SiOHR¹₂)—O—(SiR²₂—O—)ₓ—(SiOHR¹₂) (Si8-I).

Branched dimethiconols can be represented by the structural formula (Si8-II):

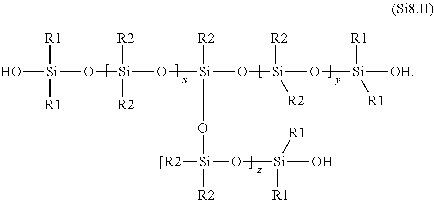

Residues R¹ and R² denote, mutually independently in each case, hydrogen, a methyl residue, a C2 to C30 linear, saturated or unsaturated hydrocarbon residue, a phenyl residue, and/or an aryl residue. The numbers x, y, and z are integers and range, mutually independently in each case, from 0 to 50,000. The molecular weights of the dimethiconols are between 1000 D and 10,000,000 D. The viscosities are between 100 and 10,000,000 cPs, measured at 25° C. using a glass capillary viscometer in accordance with Dow Corning Corporate Test Method CTM 0004 of Jul. 20, 1970. Preferred viscosities are between 1000 and 5,000,000 cPs; very particularly preferred viscosities are between 10,000 and 3,000,000 cPs. The most preferred range is between 50,000 and 2,000,000 cPs.

The following commercial products are recited as examples of such products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (all four aforesaid GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforesaid Wacker-Chemie GmbH).

Dimethiconols (Si8) are in the compositions according to the present invention in quantities from 0.01 to 10 wt %, preferably 0.01 to 8 wt %, particularly preferably 0.1 to 7.5 wt %, and in particular 0.1 to 5 wt % dimethiconol, based on the composition.

Protein hydrolysates and/or derivatives thereof (P) are a further synergistic active agent according to the present invention in the compositions according to the present invention having the active-agent complex according to the present invention.

Protein hydrolysates of both vegetable and animal origin, or of marine or synthetic origin, can be used according to the present invention.

Animal protein hydrolysates are, for example, protein hydrolysates of elastin, collagen, keratin, silk, and milk protein, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Also preferred according to the present invention are vegetable protein hydrolysates such as soy, almond, pea, moringa, potato, and wheat protein hydrolysates. Such products are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda), and Puricare® LS 9658 of the Laboratoires Sérobiologiques company.

Further protein hydrolysates preferred according to the present invention are of marine origin. These include, for example, collagen hydrolysates from fish or algae, as well as protein hydrolysates from mussels or pearl hydrolysates. Examples of pearl extracts according to the present invention are the commercial products Pearl Protein Extract BG® or Crodarom® Pearl.

Protein hydrolysates (P) are included in the compositions in concentrations from 0.001 wt % up to 20 wt %, preferably from 0.05 wt % up to 15 wt %, and very particularly preferably in quantities from 0.05 wt % up to 5 wt %.

The effect of the compositions according to the present invention can be further enhanced by a 2-pyrrolidinone-5-carboxylic acid and derivatives thereof (J). The sodium, potassium, calcium, magnesium, or ammonium salts, in which the ammonium ion carries, beside hydrogen, one to three C₁ to C₄ alkyl groups, are preferred. The sodium salt is very particularly preferred. The quantities employed in the agents according to the present invention are 0.05 to 10 wt % based on the total agent, particularly preferably 0.1 to 5, and in particular 0.1 to 3 wt %.

A further preferred group of ingredients of the compositions according to the present invention having the active-agent complex according to the present invention are vitamins, provitamins, or vitamin precursors.

Vitamins, provitamins, and vitamin precursors that are allocated to groups A, B, C, E, F, and H are particularly preferred.

The group of substances referred to as “vitamin A” includes retinol (vitamin A₁) as well as 3,4-didehydroretinol (vitamin A₂). β-Carotene is the provitamin of retinol. Vitamin A components that are appropriate according to the present invention are, for example, vitamin A acid and esters thereof, vitamin A aldehyde, and vitamin A alcohol, as well as sters thereof such as the palmitate and acetate. The agents according to the present invention include the vitamin A component preferably in quantities from 0.05 to 1 wt %, based on the total preparation.

Members of the vitamin B group or vitamin B complex are, among others:

Vitamin B₁ (thiamine)
Vitamin B₂ (riboflavin)
Vitamin B₃. The compounds nicotinic acid and nicotinic acid amide (niacinamide) are often listed under this designation. Nicotinic acid amide is preferred according to the present invention and is included in the agents used according to the present invention preferably in quantities from 0.05 to 1 wt % based on the total agent.
Vitamin B₅ (pantothenic acid, panthenol, and pantolactone). In the context of this group, panthenol and/or pantolactone are preferably used. Derivatives of panthenol that are usable according to the present invention are in particular esters and ethers of panthenol as well as cationically derivatized panthenols. Individual representatives are, for example, panthenol triacetate, panthenol monoethyl ether and the monoacetate thereof, and cationic panthenol derivatives. Pantothenic acid is used in the present invention preferably as a derivative in the form of the more stable calcium salts and sodium salts (calcium pantothenate, sodium pantothenate).
Vitamin B₆ (pyridoxine as well as pyridoxamine and pyridoxal).

The aforementioned compounds of the vitamin B type, in particular vitamin B₃, B₅, and B₆, are included in the agents according to the present invention preferably in quantities from 0.05 to 10 wt % based on the total agent. Quantities from 0.1 to 5 wt % are particularly preferred.

Vitamin C (ascorbic acid). Vitamin C is employed in the agents according to the present invention preferably in quantities from 0.1 to 3 wt % based on the total agent. Utilization in the form of the palmitic acid ester, glucosides, or phosphates can be preferred. Utilization in combination with tocopherols can likewise be preferred.

Vitamin E (tocopherols, in particular α-tocopherol). Tocopherol and its derivatives, which include in particular esters such as the acetate, nicotinate, phosphate, and succinate, are included in the agents according to the present invention preferably in quantities from 0.05 to 1 wt % based on the total agent.

Vitamin F. The term “vitamin F” is usually understood to mean essential fatty acids, in particular linoleic acid, linolenic acid, and arachidonic acid.

Vitamin H. “Vitamin H” refers to the compound (3aS,4S,6aR)-2-oxohexahydrothienol[3,4-d]imidazole-4-valeric acid, for which the trivial name “biotin” has, however, now become established. Biotin is included in the agents according to the present invention preferably in quantities from 0.0001 to 1.0 wt %, in particular in quantities from 0.001 to 0.01 wt %.

The compositions according to the present invention preferably include vitamins, provitamins, and vitamin precursors from groups A, B, E, and H. Panthenol, pantolactone, pyridoxine and derivatives thereof, as well as nicotinic acid amide and biotin, are particularly preferred.

A particularly preferred group of ingredients in the cosmetic compositions according to the present invention are the betaines recited as follows: carnitine, carnitine tartrate, carnitine magnesium citrate, acetylcarnitine, betalaines, 1,1-dimethylproline, choline, choline chloride, choline bitartrate, choline dihydrogen citrate, and the compound N,N,N-trimethylglycine referred to in the literature as “betaine.”

It is preferred to use carnitine, histidine, choline, and betaine. In a particularly preferred embodiment of the invention, L-camitine tartrate is used as an active agent.

A particularly essential ingredient is taurine and/or a derivative of taurine. “Taurine” is understood exclusively as 2-aminoethanesulfonic acid, and a “derivative” as the explicitly recited derivatives of taurine. The derivatives of taurine are understood as N-monomethyl taurine, N,N-dimethyl taurine, taurine lysylate, taurine tartrate, taurine ornithate, lysyl taurine, and ornithyl taurine. Further taurine derivatives for purposes of the present invention are taurocholic acid and hypotaurine.

Agents according to the present invention that contain, based on their weight, 0.0001 to 10.0 wt %, preferably 0.0005 to 5.0 wt %, particularly preferably 0.001 to 2.0 wt %, and in particular 0.001 to 1.0 wt % taurine and/or a derivative of taurine, are particularly preferred.

In a further embodiment preferred according to the present invention, the compositions according to the present invention include bioquinones. In agents according to the present invention, “suitable bioquinones” are to be understood as one or more ubiquinone(s) and/or plastoquinone(s). The ubiquinones preferred according to the present invention have the following formula:

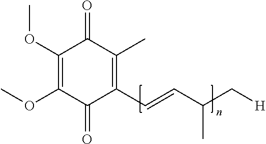

where n=6, 7, 8, 9, or 10.

Coenzyme Q-10 is most preferred in this context.

Preferred compositions according to the present invention include purine and/or purine derivatives within narrower quantitative ranges. Cosmetic agents preferred according to the present invention are characterized here in that they contain, based on their weight, 0.001 to 2.5 wt %, preferably 0.0025 to 1 wt %, particularly preferably 0.005 to 0.5 wt %, and in particular 0.01 to 0.1 wt % purine(s) and/or purine derivative(s). Cosmetic agents preferred according to the present invention are characterized in that they include purine, adenine, guanine, uric acid, hypoxanthine, 6-purinethiol, 6-thioguanine, xanthine, caffeine, theobromine, or theophylline. In hair-cosmetic preparations, caffeine is most preferred.

In a further preferred embodiment of the present invention the cosmetic agent includes ectoin ((S)-2-methyl-1,4,5,6-tetrahydro-4-pyrimidinecarboxylic acid).

Agents that contain, based on their weight, 0.00001 to 10.0 wt %, preferably 0.0001 to 5.0 wt %, and in particular 0.001 to 3 wt % active agents from the group constituted by carnitine, coenzyme Q-10, ectoin, a vitamin of the B series, a purine, and derivatives or physiologically acceptable salts thereof, are particularly preferred according to the present invention.

In a further embodiment the agents according to the present invention should additionally include at least one UV light protection filter. UVB filters can be oil-soluble or water-soluble.

The following are to be recited, for example, as oil-soluble substances:
3-benzylidene camphor, e.g. 3-(4-methylbenzylidene) camphor,
4-aminobenzoic acid derivatives, preferably 4-(dimethylamino)benzoic acid 2-ethylhexyl ester, 4-(dimethylamino)benzoic acid octyl ester, and 4-(dimethylamino)benzoic acid amyl ester,
esters of cinnamic acid, preferably 4-methoxycinnamic acid 2-ethylhexyl ester, 4-methoxycinnamic acid propyl ester, 4-methoxycinnamic acid isoamyl ester, 2-cyano-3-phenylcinnamic acid 2-ethylhexyl ester (octocrylene),
esters of salicylic acid, preferably salicylic acid 2-ethylhexyl ester, salicylic acid 4-isopropylbenzyl ester, salicylic acid homomenthyl ester,
derivatives of benzophenone, preferably 2-hydroxy-4-methoxybenzophenone, 2-hydroxy-4-methoxy-4′-methylbenzophenone, 2,2′-dihydroxy-4-methoxybenzophenone,
esters of benzalmalonic acid, preferably 4-methoxybenzalmalonic acid di-2-ethylhexyl ester,
triazine derivatives, for example 2,4,6-trianilino-(p-carbo-2′-ethyl-1′-hexyloxy)-1,3,5-triazine and octyltriazone,
propane-1,3-diones, for example 1-(4-tert-butylphenyl)-3-(4′-methoxyphenyl)propane-1,3-dione.

Suitable water-soluble substances are:
2-phenylbenzimidazole-5-sulfonic acid and alkali, alkaline-earth, ammonium, alkylammonium, alkanolammonium, and glucammonium salts thereof,
sulfonic acid derivatives of benzophenones, preferably 2-hydroxy-4-methoxybenzophenone-5-sulfonic acid and salts thereof,
sulfonic acid derivatives of 3-benzylidene camphor, for example 4-(2-oxo-3-bornylidenemethyl)benzenesulfonic acid and 2-methyl-5-(2-oxo-3-bomylidene)sulfonic acid and salts thereof.

Typical UV-A filters that are suitable are, in particular, derivatives of benzoylmethane, for example 1-(4′-tert-butylphenyl)-3-(4′-methoxyphenyl)propane-1,3-dione or 1-phenyl-3-(4′-isopropylphenyl)propane-1,3-dione. The UV-A and UV-B filters can of course also be used in mixtures. Besides the soluble substances recited, insoluble pigments are also suitable for this purpose, in particular finely dispersed metal oxides or salts, for example titanium oxide, zinc oxide, iron oxide, aluminum oxide, cerium oxide, zirconium oxide, silicates (talc), barium sulfate, and zinc stearate. The particles should have an average diameter of less than 100 nm, preferably between 5 and 50 nm, and in particular between 15 and 30 nm. They can have a spherical shape, but those particles which possess an ellipsoidal shape or one otherwise deviating from a spherical form can also be used.

Lastly, the use of plant extracts (L) in the compositions according to the present invention yields further advantages. Preferred above all according to the present invention are the extracts from green tea, oak bark, stinging nettle, witch hazel, hops, henna, chamomile, burdock, horsetail, whitethorn, linden blossom, almond, aloe vera, spruce needles, horse chestnut, sandalwood, juniper, coconut, mango, apricot, lemon, wheat, kiwi, melon, orange, grapefruit, sage, rosemary, birch, mallow, valerian, lady's smock, wild thyme, yarrow, thyme, lemon balm, restharrow, coltsfoot, hibiscus, meristem, ginseng, coffee, cocoa, moringa, ginger root, and Ayurvedic plant extracts such as Aegle marmelos (bilwa), Cyperus rotundus (nagar motha), Emblica officinalis (amalki), Morida citrifolia (ashyuka), Tinospora cordifolia (guduchi), Santalum album (chandana), Crocus sativus (kumkuma), Cinnamonum zeylanicum, and Nelumbo nucifera (kamala), sweet grasses such as wheat, barley, rye, oats, spelt, corn, the various types of millet (proso millet, finger millet, foxtail millet as examples), sugar cane, ryegrass, meadow foxtail, false oat-grass, bentgrass, meadow fescue, moor grass, bamboo, cottongrass, pennisetums, Andropogonodeae (Imperata cylindrica, also known as blood grass or cogon grass), buffalo grass, cord grass, dog's tooth grass, lovegrass, Cymbopogon (citronella grass), Oryzeae (rice), Zizania (wild rice), marram grass, blue oatgrass, soft-grasses, quaking grasses, speargrasses, couch grasses and Echinacea, in particular Echinacea angustifolia DC, Echinacea paradoxa (Norton), Echinacea simulata, E. atrorubens, E. tennesiensis, Echinacea strigosa (McGregor), Echinacea laevigata, Echinacea purpurea (L.) Moench and Echinacea pallida (Nutt), all types of vine, and pericarp of Litchi chinensis.

The plant extracts can be used according to the present invention in both pure and dilute form. If they are used in dilute form, they usually include approx. 2 to 80 wt % active substance and, as a solvent, the extraction agent or extraction agent mixture used to recover them.

The cosmetic agents can additionally include further active agents, adjuvants, and additives, for example:
structuring agents such as maleic acid and lactic acid,
swelling agents such as urea, allantoin, carbonates, or hydantoin,
dimethylisosorbide and cyclodextrins,
dyes for coloring the agent,
anti-dandruff active agents such as piroctone olamine, zinc omadine, and climbazole,
complexing agents such as EDTA, NTA, β-alaninediacetic acid, and phosphonic acids, opacifiers such as latex, styrene/PVP copolymers and styrene/acrylamide copolymers,
luster agents such as ethylene glycol mono- and distearate as well as PEG-3 distearate,
pigments,
stabilizing agents for hydrogen peroxide and other oxidizing agents,
propellants such as propane/butane mixtures, N₂O, dimethyl ether, CO₂, and air,
antioxidants
perfume oils, scents, and fragrances.

With regard to further optional components as well as the quantities of those components used, reference is made expressly to the relevant manuals known to one skilled in the art.

As already mentioned, the excellent care-providing effect of the agents according to the present invention is of particular significance. Preferred agents according to the present invention contain, besides the highly preferred components of the active-agent complex, in particular behentrimonium chloride, stearamidopropyl dimethylamine, isopropyl myristate and dicaprylyl carbonate, Polyquaternium-37, and lauryl glucoside, furthermore a cationic aminofunctional silicone, highly preferably Silicone Quaternium-22, a vitamin of the B series, and additionally at least one of the following active agents (i) to (viii):
(i) at least taurine or at least one derivative of taurine,
(ii) at least one purine, in particular caffeine,
(iii) at least carnitine,
(iv) at least ectoin,
(v) at least one ubiquinone, in particular coenzyme Q-10,
(vi) at least one UV absorption agent,
(vii) at least one plant extract, selected in particular from extracts of hops, ginseng, Litchi chinensis, or Echinacea,
(viii) ectoin.

A further subject of the invention is use of the composition according to the present invention to improve the robustness of the surface of keratinic fibers with respect to physical damage. “Physical damage” is understood according to the present invention as the action of UV light, the action of heat while blow-drying, mechanical actions when combing and brushing the hair.

A further subject of the present invention is use of the composition according to the present invention to improve the wash-out resistance of colored keratinic fibers.

A further subject of the invention is a method for hair treatment, in which method a cosmetic agent according to claim 1 is applied onto the hair and rinsed out of the hair after a contact time.

The contact time is preferably from a few seconds to 100 minutes, particularly preferably 1 to 50 minutes, and very particularly preferably 1 to 30 minutes.

Also in accordance with the invention is a method in which a cosmetic agent according to claim 1 is applied onto the hair and remains there. “Remains on the hair” is understood according to the present invention to mean that the agent is not rinsed out of the hair again immediately after it is applied. In this case the agent instead remains on the hair for more than 100 minutes, until the hair is next washed.

### Examples

Unless otherwise indicated, all quantity indications are parts by weight. The following formulas were provided using known manufacturing methods.

TABLE 1
Test compositions

| Ingredient | Comparison 1 | Inventive 1 (I1) |
|---|---|---|
| Cetearyl Alcohol | 5.0 | 2.7 |
| Distearoylethyl Hydroxyethylmonium | 1.0 | — |
| Methosulfate | | |
| Ceteareth-20 | 0.3 | — |
| Cetrimonium chloride | 4.0 | — |
| Amodimethicone | 1.0 | — |
| Amodimethicone/Morpholinomethyl | — | 1.0 |
| Silsesquioxane Copolymer | | |
| Behentrimonium chloride | — | 1.8 |
| Stearamidopropyl dimethylamine | — | 0.4 |
| Glyceryl monostearate | 0.3 | 0.3 |
| Paraffin Liquidum | 0.8 | — |
| Isopropyl myristate | — | 0.8 |
| Citric acid | 0.45 | 0.5 |
| Methylparaben sodium salt | 0.3 | 0.3 |
| Polyquaternium-37 | — | 0.2 |
| Dicaprylyl carbonate | — | 0.15 |
| Lauryl glucoside | — | 0.01 |
| Panthenol | 0.2 | 0.2 |
| Nicotinic acid amide | 0.15 | 0.15 |
| Phenoxyethanol | 0.4 | 0.4 |
| Perfume 07 | 0.3 | 0.3 |
| Water | to 100 | to 100 |
| pH | 2.5 | 2.5 |

In an independent testing institute for the cosmetic industry, both formulas were tested on 20 women aged between 28 and 67 years using the “half head” test. Hair characteristics—hair lengths, hair thickness, greasiness, and prior damage—were mixed. This means that all characteristics were uniformly represented. After evaluation, the parameters of washability, distributability, skin feel, skin feel after rinsing, wet hair feel, detangling, wet hair combability, wet hair softness, curl retention behavior, hair volume, antistatic properties, and handling were appreciably improved. All the improved properties observed were statistically significant.

In a second series of tests, the following compositions were tested in the half head test as already described above.

TABLE 2
Compositions of test formulas (C = comparison; I = inventive)

| Ingredient | C2 | C3 | C4 | C5 | C6 | I2 |
|---|---|---|---|---|---|---|
| Cetearyl Alcohol | 2.5 | 2.5 | 2.5 | 2.5 | 2.5 | 2.5 |
| Paraffin Liquidum | 0.6 | 0.6 | 0.6 | 0.6 | 0.6 | 0.6 |
| Glyceryl monostearate | 0.3 | 0.3 | 0.3 | 0.3 | 0.3 | 0.3 |
| Dicaprylyl carbonate | 0.16 | 0.16 | 0.16 | 0.16 | 0.16 | 0.16 |
| Methylparaben Na salt | 0.2 | 0.2 | 0.2 | 0.2 | 0.2 | 0.2 |
| Citric acid | 0.25 | 0.25 | 0.25 | 0.25 | 0.25 | 0.25 |
| Phenoxyethanol | 0.4 | 0.4 | 0.4 | 0.4 | 0.4 | 0.4 |
| Perfume | 0.3 | 0.3 | 0.3 | 0.3 | 0.3 | 0.3 |
| Trideceth-5 | 0.125 | 0.125 | 0.125 | 0.125 | 0.125 | 0.125 |
| Behentrimonium chloride | 0.5 | 0.5 | 0.5 | 0.5 | 0.5 | 0.5 |
| Isopropyl myristate | 0.6 | 0.6 | 0.6 | 0.6 | 0.6 | 0.6 |
| Stearamidopropyl | — | — | 0.4 | 0.4 | 0.4 | 0.4 |
| dimethylamine | | | | | | |
| Amodimethicone/ | — | 0.5 | — | 0.5 | — | 0.5 |
| Morpholinomethyl | | | | | | |
| Silsesquioxane Copolymer | | | | | | |
| Polyquaternium-37 | — | — | — | 0.2 | 0.2 | 0.2 |
| Lauryl glucoside | — | — | — | — | 0.01 | 0.01 |

| | |
|---|---|
| Water | to 100 |
| pH | 2.5 |

TABLE 3
Results of half-head test

Scoring

| Test criterion | C2 | C3 | C4 | C5 | C6 | I2 |
|---|---|---|---|---|---|---|
| Wet hair feel | 0 | 0.3 | 0.5 | 0.8 | 0.8 | 1.5 |
| Wet hair combability | 0 | 0.5 | 0.5 | 0.6 | 0.7 | 1.8 |
| Detangling | 0 | 0 | 0.6 | 0.7 | 0.4 | 1.3 |
| Hairstyle volume | 0 | 0.4 | 0.3 | 0.4 | 0.4 | 1.7 |
| Antistatic properties | 0 | 0.4 | 0.3 | 0.5 | 0.5 | 1.6 |
| Dry hair feel | 0 | 0.5 | 0.5 | 0.5 | 0.3 | 1.6 |
| Dry hair combability | 0 | 0.3 | 0.5 | 0.5 | 0.5 | 1.7 |

The table lists the significant differences. The assessment was made in each case using 20 women aged between 28 and 70 years. Hair characteristics—hair lengths, hair thickness, greasiness, and prior damage—were mixed. This means that all characteristics were uniformly represented. The evaluation was carried out by five trained hairdressers. The scores assigned in each case were added, and the arithmetic mean is reproduced in the table as an evaluation of the respective parameter relative to the standard (C2). Because C2 is considered the standard against which evaluation was made, the standard was set to zero. A higher score compared with the standard thus means a better evaluation, and the score indicates the difference from the standard.

Compositions C3 to C6 were all evaluated as better than composition C2. Composition C2 corresponds to a usual standard conditioner. In compositions C3 to C6, various additives are added in order to improve the properties of a conditioner. These show a slight improvement in performance as compared with composition C2. Composition 12 according to the present invention exhibits a surprisingly appreciable improvement in performance values. This is surprising and unexpected, since the addition of individual or multiple ingredients (see C3 to C6) that are each individually known to be conditioning does not contribute to an appreciable improvement compared with C2. The results are statistically significant in accordance with the Student p-test.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for conditioning keratinic fibers, including, in a cosmetic carrier,
a) 0.1 to 6.0 wt % of at least one cationic surfactant selected from the group consisting of behentrimonium chloride, dimethyldibehenylammonium chloride, tribehenylmethyltrimonium chloride, cetyltrimethylammonium chloride, dicetyldimethylammonium chloride, tricetylmethylammonium chloride, trimethylstearylammonium chloride, dimethyldistearylammonium chloride, and tristearylmethylammonium chloride,
b) 0.01 to 6.0 wt % of at least one amidoamine and/or one permanently cationic amidoamine,
c) 0.01 to 7.5 wt % of at least one ester oil,
d) 0.01 to 5.0 wt % of at least one cationic polymer selected from the group consisting of Polyquaternium-67, Polyquaternium-72, Polyquaternium-74, and Polyquatemium-37,
e) 0.01 to 10.0 wt % of at least one amodimethicone selected from the group consisting of amodimethicones having the INCI name amodimethicone/morpholinomethyl silsesquioxane copolymer, and
f) 0.001 to 3.0 wt % of at least one sugar surfactant,
all quantity indications being based on the total weight of the cosmetic composition.

2. The cosmetic composition according to claim 1, wherein the ester oil is selected from symmetrical, asymmetrical, or cyclic esters of carbonic acid with fatty alcohols.

3. The cosmetic composition according to claim 1, wherein the sugar surfactant is a C8 to C18 alkyl oligoglucoside.

4. The cosmetic composition according to claim 1, wherein the cationic polymer is Polyquatemium-37.

5. The cosmetic composition according to claim 1, wherein the cationic surfactant is behentrimonium chloride or cetyltrimonium chloride.

6. The cosmetic composition according to claim 1, wherein the ester oil is dicaprylyl carbonate.

7. The cosmetic composition according to claim 1, further including a vitamin of the B series.

8. A method for improving the robustness of the surface of keratinic fibers, including:
applying a composition according to claim 1 onto washed and towel-dried keratinic fibers for a time from a few seconds to 10 minutes, and
rinsing the composition out of the keratinic fibers.
